(19) United States
(12) Patent Application Publication
(10) Pub. No.: US 2025/0195426 A1
(43) Pub. Date: Jun. 19, 2025

(54) HIGH DRUG-LOADING FULVESTRANT PHARMACEUTICAL COMPOSITION AND PREPARATION METHOD THEREOF

(71) Applicant: Qilu Pharmaceutical Co., LTD., Jinan (CN)

(72) Inventors: Yong ZHANG, Jinan (CN); Donghai WANG, Jinan (CN); Qingmin YANG, Jinan (CN); Ming WANG, Jinan (CN)

(73) Assignee: Qilu Pharmaceutical Co., LTD., Jinan (CN)

(21) Appl. No.: 18/821,393

(22) Filed: Aug. 30, 2024

(30) Foreign Application Priority Data
Dec. 13, 2023 (CN) 202311724041.0

(51) Int. Cl. A61K 9/10; A61K 31/565; A61K 47/02; A61K 47/10; A61K 47/12; A61K 47/26

(52) U.S. Cl. CPC A61K 9/10; A61K 31/565; A61K 47/02; A61K 47/10; A61K 47/12; A61K 47/26

(57) ABSTRACT

The present disclosure provides a high drug-loading fulvestrant pharmaceutical composition and a preparation method thereof, and the pharmaceutical composition comprises an active ingredient, an excipient, and an aqueous carrier, free of ethanol and an oily substrate, wherein the active ingredient is fulvestrant or an ester derivative of fulvestrant, the excipient comprises one or more selected from the group consisting of a wetting agent, a suspending agent, a buffer agent, and an osmotic pressure regulator, the active ingredient is dispersed in the aqueous carrier, the excipient is dissolved in the aqueous carrier, and a concentration of the active ingredient is from 150 mg/ml to 550 mg/ml, calculated on the basis of fulvestrant itself. The pharmaceutical composition of the present disclosure has a high concentration of the drug, and can effectively reduce the dosing volume to about 2 ml-8 ml, and achieve an efficient drug delivery for high-dose treatment of patients. The pharmaceutical composition of the present disclosure can be administered by intramuscular injection to obtain a sustained release of the drug for up to 1-3 months, thereby effectively improving the patient compliance.

### TECHNICAL FIELD

The present disclosure belongs to the technical field of pharmaceutical preparations, and specifically relates to a high drug-loading fulvestrant pharmaceutical composition and a preparation method thereof.

### BACKGROUND

Fulvestrant has a structural formula as follows:

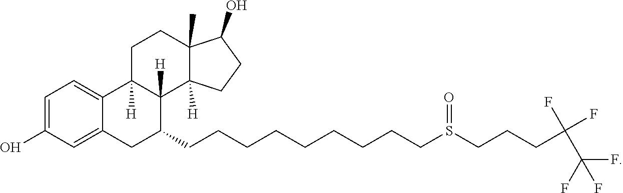

Fulvestrant injection was approved for marketing by the US FDA in 2002, and is an anti-estrogen type therapeutic drug for treating breast cancer, a selective down-regulator of an estrogen receptor, and a specific antagonist of an estrogen receptor. Since the estrogen receptor is found in many breast cancer patients and tumor growth is stimulated by estrogen, the current primary treatment for breast cancer is to reduce the concentration of estrogen. Fulvestrant not only can compete with the estrogen receptor for binding, but also can block the receptor, inhibit the binding of estrogen, stimulate morphological changes in the receptor, reduce estrogen receptor concentration, and damage tumor cells. Fulvestrant minimizes tumor growth by down-regulating estrogen receptor proteins in human breast cancer cells.

The currently marketed fulvestrant injection (Faslodex) is a solution-type injection in a non-aqueous solvent with a specification of 5 ml:250 mg, a drug loading capacity of only 50 mg/mL, and an active ingredient content/concentration of only 5%. The clinical dosage of the commercially available fulvestrant injection is 500 mg, which is administered by intramuscular injection, with 250 mg of the drug to be injected into each of the left and right buttocks each time, i.e., 5 ml of the drug to be injected on each side, for a total injection of 10 mL. Patient compliance is poor due to the large dosing volume and high dosing frequency, and the need for the patients to endure pain twice. In addition, the commercially available fulvestrant injection comprises an irritating excipient, such as ethanol, castor oil, benzyl benzoate, and so on. The intramuscular injection of fulvestrant is highly painful and prone to cause injection-related adverse effects, including myositis and granuloma at injection sites.

Patent publication No. CN103070871A discloses a pharmaceutical composition of fulvestrant, wherein the concentration of fulvestrant can reach 45-115 mg/ml. However, the composition comprises ethanol, benzyl alcohol, medium-chain triglyceride, or castor oil as a matrix, which belongs to a solution-type non-aqueous carrier with related adverse effects such as intramuscular injection irritation and others. Patent publication No. CN111481559A discloses a pharmaceutical composition with a fulvestrant loading amount of up to 10% and comprising a solution-type non-aqueous carrier. Although benzyl alcohol and/or benzyl benzoate are/is not used in the composition, irritating excipients, such as ethanol and castor oil, are still used to improve the solubility of fulvestrant. Patent publication No. CN111035613A also discloses a high drug-loading pharmaceutical composition of fulvestrant comprising a solution-type carrier, in which benzyl alcohol, benzyl benzoate, and castor oil are not used, but an organic solvent, such as ethanol and a medium-chain triglyceride, is still used to improve the solubility of fulvestrant. None of the above patent publications address the use of ethanol and an oily non-aqueous solvent.

Patent publication No. CN107789320A discloses a sustained-release injection of fulvestrant free of an oily solvent. For a sustained-release suspension, increasing the drug loading capacity is a technical challenge for this type of preparation. In this patent publication, the drug loading capacity is only increased to at most 100 mg/ml, and the dosing interval is only 10-15 days/dose. Patent publication No. CN111479556A discloses an aqueous suspension of fulvestrant comprising both dissolved fulvestrant and undissolved fulvestrant, wherein the drug loading capacity is increased to about 100 mg/ml by the two forms of fulvestrant, but the suspension still fails to solve the problem of single-site administration of fulvestrant in large doses for intramuscular injection, and still needs to injected at multiple sites.

Fulvestrant is almost insoluble in water, thereby resulting in its unstable or incomplete absorption in vivo and poor bioavailability, and limiting the drug efficacy. How to increase the solubility, dissolution rate, and bioavailability of fulvestrant to achieve a prolonged steady release of fulvestrant is an urgent problem to be solved.

Solubilization is mainly carried out by ethanol or an oily solvent in the prior art. This technique is difficult to obtain a high drug loading capacity, and has a muscle irritation problem caused by an organic solvent such as ethanol. There is also a technique to increase the solubility of fulvestrant by controlling the particle size of the drug, but this technique is less likely to result in a high drug loading capacity and sill fails to solve the problem of single-site injection of fulvestrant in large doses. Therefore, how to obtain a fulvestrant injection having at least one of a high drug loading capacity, small dosing volume, long drug release period, good patient compliance, high drug safety, small fluctuation of the drug concentration in the body, and steady drug efficacy without using an organic solvent is a technical problem to be solved in the present disclosure.

### SUMMARY

In one aspect, the present disclosure provides a pharmaceutical composition comprising an active ingredient, an excipient, and an aqueous carrier, and free of ethanol and an oily matrix, wherein the active ingredient is selected from fulvestrant or an ester derivative of fulvestrant; the excipient comprises one or more selected from the group consisting of a wetting agent, a suspending agent, a buffer agent, and an osmotic pressure regulator; and a concentration of the active ingredient is from 150 mg/ml to 550 mg/ml, preferably from 200 mg/ml to 400 mg/ml, calculated on the basis of fulvestrant itself.

In another aspect, the present disclosure provides a method for preparing the pharmaceutical composition of present disclosure by air jet pulverization, high-speed shearing, media grinding, or high-pressure homogenization.

In yet another aspect, the present disclosure provides a method for treating advanced or metastatic breast cancer in a patient in need thereof, comprising administering the pharmaceutical composition of the present disclosure to the patient.

### DETAILED DESCRIPTION

In order to solve the above-mentioned problems in the prior art, the present disclosure provides a pharmaceutical composition of fulvestrant with at least one of a high drug-loading capacity, small dosing volume, safe and stable quality, long sustained-release period, simple preparation process, and adaptability to large-scale production, and a preparation method thereof.

In one aspect, the present disclosure provides a pharmaceutical composition comprising an active ingredient, an excipient, and an aqueous carrier, and free of ethanol and an oily matrix, wherein the active ingredient is selected from fulvestrant or an ester derivative of fulvestrant; the excipient comprises one or more selected from the group consisting of a wetting agent, a suspending agent, a buffer agent, and an osmotic pressure regulator; and a concentration of the active ingredient is from 150 mg/ml to 550 mg/ml, preferably from 200 mg/ml to 400 mg/ml, calculated on the basis of fulvestrant itself.

In the pharmaceutical composition of the present disclosure, the active ingredient is dispersed in the aqueous carrier, and the excipient is dissolved in the aqueous carrier.

The pharmaceutical composition of the present disclosure can form a drug reservoir with a drug amount of from about 500 mg to 1,500 mg at an injection site. Preferably, the drug amount is from 500 mg to 750 mg, or from 750 mg to 1,000 mg, or from 1,000 mg to 1,250 mg, or from 1,250 mg to 1,500 mg.

Fulvestrant has a larger effective dosage, and is currently administered at a clinical dosage of 500 mg. The inventors of the present disclosure have found through researches that too low a concentration of the drug (active ingredient) can lead to too large an injection volume and poor patient compliance, and too high a concentration of the drug can lead to clogging of an injection needle or aggregation of the drug particles during storage, making injection difficult. Therefore, the above concentration range selected in the present disclosure is an optimal concentration range based on the high concentration of the drug. With the higher drug concentration of the present disclosure, the dosing dosage can be increased to 1,500 mg.

The pharmaceutical composition of the present disclosure is administered in a volume of from 2 ml to 8 ml, preferably from 3 ml to 6 ml.

The pharmaceutical composition of the present disclosure comprises the active ingredient and the excipient at the following contents in weight percentage (w/w):

| | |
|---|---|
| active ingredient | 15-55% |
| wetting agent | 0.1-2% |
| suspending agent | 0.1-20% |
| buffer agent | 0.1-10% |
| osmotic pressure regulator | 0.1-15% |
| aqueous carrier | balance to 100%. |

In some preferred embodiments, the pharmaceutical composition of the present disclosure comprises the active ingredient and the excipient at the following contents in weight percentage (w/w):

| | |
|---|---|
| active ingredient | 20-40% |
| wetting agent | 0.3-1.5% |
| suspending agent | 0.3-16% |
| buffer agent | 0.3-5% |
| osmotic pressure regulator | 0.3-10% |
| aqueous carrier | balance to 100%. |

The aqueous carrier in the pharmaceutical composition of the present disclosure is selected from water for injection, glucose injection, or normal saline. In some preferred embodiments, the aqueous carrier is water for injection.

The excipient in the pharmaceutical composition of the present disclosure comprises one or more selected from the group consisting of a wetting agent, a suspending agent, a buffer agent, an osmotic pressure regulator, an optional additional component, and an aqueous carrier.

In some preferred embodiments, the wetting agent in the pharmaceutical composition of the present disclosure is selected from one or more of polysorbate 20, polysorbate 60, polysorbate 80, lecithin, sodium deoxycholate, poloxamer, and sodium lauryl sulfate.

In some preferred embodiments, the suspending agent in the pharmaceutical composition of the present disclosure is selected from one or more of polyethylene glycol 4000, polyethylene glycol 6000, sodium carboxymethylcellulose, hypromellose, hydroxypropylcellulose, and povidone.

In some preferred embodiments, the buffer agent in the pharmaceutical composition of the present disclosure is selected from one or more of phosphate, citric acid or citrate, tartaric acid or tartrate, acetate, HEPES, Tirs, and sodium hydroxide.

In some preferred embodiments, the osmotic pressure regulator in the pharmaceutical composition of the present disclosure is selected from one or more of sucrose, mannitol, trehalose, sodium chloride, phosphate, citric acid or citrate, and tartaric acid or tartrate.

In some preferred embodiments, the active ingredient in the pharmaceutical composition of the present disclosure has an average specific surface area of 0.5-5.8 m²/g. After intramuscular injection, the pharmaceutical composition of the present disclosure can sustainedly release the active ingredient for up to 30 days or more.

In other preferred embodiments, the active ingredient in the pharmaceutical composition of the present disclosure has an average specific surface area of 0.5-2.5 m²/g. After intramuscular injection, the pharmaceutical composition of the present disclosure can sustainedly release the active ingredient for up to 60 days or more.

In still other preferred embodiments, the active ingredient in the pharmaceutical composition of the present disclosure has an average specific surface area of 0.5-0.9 m²/g. After intramuscular injection, the pharmaceutical composition of the present disclosure can sustainedly release the active ingredient for up to 90 days or more.

Therefore, the drug of the present disclosure can be released slowly for 1 month or more at the above-mentioned particular specific surface area of 0.5-5.8 m²/g, thereby reducing the injection frequency of the drug. In some preferred embodiments of the present disclosure, only one injection is required every month, or only one injection is required every two months, or only one injection is required every three months.

The pharmaceutical composition of the present disclosure greatly improves the stability of the drug and can be stable at room temperature for more than 12 months by controlling the specific surface area of the active ingredient to the above-mentioned range, compared with the organic solvent-based preparations in the prior art.

The larger the specific surface area of the active ingredient in the pharmaceutical composition of the present disclosure, the faster the drug release, and the smaller the specific surface area, the slower the drug release. As described above, the pharmaceutical composition of the present disclosure can be released for about 1-3 months after intramuscular injection into the buttocks, and the injection volume can be reduced to about 2-8 ml compared with the preparations in the prior art.

In some preferred embodiments, the pharmaceutical composition of the present disclosure is in the form of a suspension. In other preferred embodiments, the pharmaceutical composition of the present disclosure is in the form of a blocky or powdered composition converted by lyophilization, which is reconstituted into a suspension using water for injection, glucose injection, or normal saline at the time of clinical use, and fulvestrant in the resulting suspension is at a concentration of 150 mg/ml-550 mg/ml, preferably 200 mg/ml-400 mg/ml.

In another aspect, the present disclosure provides a method for preparing the pharmaceutical composition in any one of the above embodiments in the form of a pharmaceutical suspension by air jet pulverization (method I), comprising the following steps:
(1) air jet pulverizing the active ingredient to the desired average specific surface area to obtain the micronized active ingredient, and
(2) dissolving the excipient in the aqueous carrier, adding the micronized active ingredient, and dispersing the components uniformly by stirring or shearing to obtain the pharmaceutical suspension.

In a preferred embodiment, the desired average specific surface area in step (1) of the method I is 0.5-5.8 m²/g.

In a preferred embodiment, the “air jet pulverization” in step (1) of the method I is carried out by using an air jet pulverizer and adjusting the pulverization pressure to between 0.1 Mpa and 1.0 Mpa.

In a preferred embodiment, the “stirring or shearing” in step (2) of method I is carried out at a revolving speed of the machine preferably adjusted to between 300 rpm and 30,000 rpm, and the pharmaceutical suspension is maintained at a temperature of 0-50° C.

In another aspect, the present disclosure also provides a method for preparing the pharmaceutical composition in any one of the above embodiments in the form of a pharmaceutical suspension by high-speed shearing, media grinding, or high-pressure homogenization (method II), comprising the following steps:
(1) dissolving the excipient in the aqueous carrier by stirring, then adding the active ingredient, and dispersing the active ingredient uniformly by stirring or preliminary shearing to obtain a primary pharmaceutical suspension, and
(2) subjecting the primary pharmaceutical suspension to high-speed shearing, media grinding, or high-pressure homogenization, so that the active ingredient has a specific surface area of 0.5-5.8 m²/g, thereby obtaining the pharmaceutical composition in the form of the suspension.

In a preferred embodiment, the “stirring or preliminary shearing” in step (1) of method II is carried out at a revolving speed of the machine preferably adjusted to between 300 rpm and 30,000 rpm, and the pharmaceutical suspension is maintained at a temperature of 0-50° C.

In a preferred embodiment, the “high-speed shearing” in step (2) of method II is carried out at a revolving speed of the machine preferably adjusted to between 3,000 rpm and 60,000 rpm, and the pharmaceutical suspension is maintained at a temperature of 0-50° C.

In some preferred embodiments, the “media grinding” in step (2) of method II is carried out by using a sand mill or ball mill at a revolving speed of 500 rpm-10,000 rpm, which can be performed several times. In some preferred embodiments, the primary pharmaceutical suspension is pumped through a peristaltic pump or added directly under continuous stirring, to prevent drug precipitation. The grinding of the pharmaceutical suspension is performed at a temperature maintained between 0° C. and 50° C. In a preferred embodiment, the grinding ball is a zirconia ball with a diameter of 0.2-2.0 mm.

In some preferred embodiments, the “high-pressure homogenization” in step (2) of method II is carried out through a micro-jet high-pressure homogenizer or an ordinary high-pressure homogenizer. The micro-jet high-pressure homogenizer has a counter-jet interaction chamber of 50-500 μm, and the primary pharmaceutical suspension is pumped through a peristaltic pump or added directly under continuous stirring, to prevent drug precipitation. The micro-jet high-pressure homogenizer or ordinary high-pressure homogenizer has a pressure of 3,000 psi-60,000 psi, and the pharmaceutical suspension is maintained at a temperature of 0-50° C.

In some preferred embodiments, the preparation method of the present disclosure further comprises the following steps:

filling the pharmaceutical composition in the form of the suspension to obtain a pharmaceutical product in the form of a liquid, or lyophilizing the pharmaceutical composition in the form of the suspension to obtain a lyophilized product of fulvestrant in the form of a block or powder, which can be diluted to the required dosage with water for injection, glucose injection, or normal saline at the time of clinical use. In some preferred embodiments, the lyophilized product of fulvestrant is reconstituted into a suspension of fulvestrant at a concentration of 150 mg/ml-550 mg/ml for clinical use. In other preferred embodiments, the lyophilized product of fulvestrant is reconstituted into a suspension of fulvestrant at a concentration of 200 mg/ml-400 mg/ml for clinical use.

In yet another aspect, the present disclosure provides a method for treating advanced or metastatic breast cancer in a patient in need thereof, comprising administering the pharmaceutical composition as described above to the patient.

In yet another aspect, the present disclosure provides a use of the pharmaceutical composition as described above in the treatment of advanced or metastatic breast cancer in a patient in need thereof.

In yet another aspect, the present disclosure provides a use of the pharmaceutical composition as described above in the preparation of a medicament for treating advanced or metastatic breast cancer.

In yet another aspect, the present disclosure provides the pharmaceutical composition as described above for use in the treatment of advanced or metastatic breast cancer.

The pharmaceutical composition of the present disclosure can be used for the treatment of local advanced or metastatic breast cancer in patients who have relapsed after or during adjuvant anti-estrogen therapy, or in postmenopausal (including naturally menopausal and artificially menopausal) estrogen receptor-positive patients who have progressed during anti-estrogen therapy. The pharmaceutical composition of the present disclosure can be used in combination with abemaciclib for the treatment of hormone receptor (HR)-positive or human epidermal growth factor receptor 2 (HER2)-negative local advanced or metastatic breast cancer in patients who have experienced disease progression following previous endocrinotherapy.

The pharmaceutical composition of the present disclosure can achieve at least one of the following beneficial effects:

1. The pharmaceutical composition of the present disclosure has the fulvestrant concentration of up to from 150 mg/ml to 550 mg/ml, which effectively reduces the dosing volume to from about 2 ml to 8 ml, thereby enabling efficient drug delivery for high-dose treatment of patients, enhancing the therapeutic efficacy and improving the patient compliance.
2. The pharmaceutical composition of the present disclosure can be administered by intramuscular injection to obtain a sustained and slow release of the drug for up to 1-3 months, thereby effectively improving the patient compliance.
3. The pharmaceutical composition of the present disclosure comprises an aqueous carrier as a solvent without introducing an organic solvent, thereby reducing the toxicity of the composition, increasing the safety of the composition, and improving the patient compliance.
4. The pharmaceutical composition of the present disclosure is a long-acting microcrystalline preparation, and can remain stable for a long period of time.
5. The pharmaceutical composition of the present disclosure can be obtained by any one or more methods including air jet pulverization, high-speed shearing, media grinding, and high-pressure homogenization, and the preparation method is simple, easy to implement, and suitable for industrial production, and the resulting pharmaceutical product is stable, reliable, and easy for storage and transport.
6. The pharmaceutical composition of the present disclosure has good stability, and can be stored at room temperature.

### BRIEF DESCRIPTION OF DRAWINGS

FIG. 1 shows the pharmacokinetic profile of the pharmaceutical composition of fulvestrant in beagle dogs.

### EXAMPLE

The present disclosure will be further illustrated below with reference to specific examples. It should be understood that the examples of the present disclosure are only for the purpose of illustrating the present disclosure, and are not intended to limit the present disclosure. Technical solutions obtained by simple improvements to the present disclosure or by equivalent substitutions using conventional means or components on the basis of the technical solutions of the present disclosure are all within the protection scope of the present disclosure.

The terms used in the specification are only for the purpose of describing specific embodiments, and are not intended to limit the scope of the present disclosure. Unless otherwise defined, all technical and scientific terms used in the specification have the same meanings as commonly understood by those of ordinary skill in the art to which the present disclosure belongs.

### Example 1 Preparation of 150 mg/ml Pharmaceutical Composition by Air Jet Pulverization

| Formula | Amount | |
|---|---|---|
| Fulvestrant | 52.5 | g |
| Polysorbate 20 | 1.2 | g |
| Polyethylene glycol 4000 | 16 | g |
| Citric acid | 5 | g |
| Sodium dihydrogen phosphate | 4.2 | g |
| Sodium hydroxide | 3.8 | g |

| | |
|---|---|
| Water for injection | Balance to 350 ml |

### Preparation Process:

(1) Fulvestrant was weighted and air-jet pulverized under the pressure of 0.8 Mpa to obtain the active ingredient with the specific surface area of 0.509 m²/g.
(2) All the excipients were dissolved in water, and micronized fulvestrant was added, and then dispersed uniformly by shearing with a shearer for 5-15 min at a revolving speed of 3,000 rpm, to obtain the final pharmaceutical suspension (150 mg/ml).

The injection volume was reduced by 3 times, compared with the marketed product fulvestrant injection (50 mg/ml).

### Example 2 Preparation of 150 mg/ml Pharmaceutical Composition by High-Speed Shearing

| Formula | Amount | |
|---|---|---|
| Fulvestrant | 52.5 | g |
| Polysorbate 20 | 1.2 | g |
| Polyethylene glycol 4000 | 16 | g |
| Citric acid | 5 | g |
| Sodium dihydrogen phosphate (anhydrous) | 4.2 | g |
| Sodium hydroxide | 3.8 | g |

| | |
|---|---|
| Water for injection | Balance to 350 ml |

### Preparation Process:

(1) All the excipients were added to water, stirred and dissolved, and then fulvestrant was added and dispersed uniformly by further stirring with a stirrer at a speed of 800 rpm, to obtain a primary pharmaceutical suspension; and
(2) The primary pharmaceutical suspension was subjected to high-speed shearing with a shearer at a revolving speed of 10,000-30,000 rpm for 20-40 min, to form the final pharmaceutical suspension (150 mg/ml).

The injection volume was reduced by 3 times, compared with the marketed product fulvestrant injection (50 mg/ml).

### Example 3 Preparation of 250 mg/ml Pharmaceutical Composition by Media Grinding

| Formula | Amount | |
|---|---|---|
| Fulvestrant | 87.5 | g |
| Polysorbate 60 | 1.45 | g |
| Polyethylene glycol 6000 | 28 | g |
| Sodium tartrate | 3.0 | g |
| Sodium dihydrogen phosphate | 2.1 | g |
| Sodium acetate | 2.3 | g |

| | |
|---|---|
| Water for injection | Balance to 350 ml |

### Preparation Process:

(1) All the excipients were added to water, stirred and dissolved, and then fulvestrant was added and dispersed uniformly by preliminary shearing with a shearer at a revolving speed of 3,000 rpm, to obtain a primary pharmaceutical suspension; and
(2) The primary pharmaceutical suspension was subjected to media grinding using a sand mill at a revolving speed of 1,000-2,000 rpm and a stirrer at a stirring speed of 300 rpm, to obtain the final pharmaceutical suspension (250 mg/ml) after grinding.

The injection volume was reduced by 5 times, compared with the marketed product fulvestrant injection (50 mg/ml).

### Example 4 Preparation of 250 mg/ml Pharmaceutical Composition by High-Pressure Homogenization

| Formula | Amount | |
|---|---|---|
| Fulvestrant | 87.5 | g |
| Polysorbate 60 | 1.45 | g |
| Polyethylene glycol 6000 | 28 | g |
| Sodium tartrate | 3.0 | g |
| Sodium dihydrogen phosphate | 2.1 | g |
| HEPES | 2.3 | g |

| | |
|---|---|
| Water for injection | Balance to 350 ml |

### Preparation Process:

(1) All the excipients were added to water, stirred and dissolved, and then fulvestrant was added and dispersed uniformly by preliminary shearing with a shearer at a revolving speed of 3,000 rpm, to obtain a primary pharmaceutical suspension; and
(2) The primary pharmaceutical suspension was directly added to a micro-jet high-pressure homogenizer, and homogenized for 2-10 min; the micro-jet high-pressure homogenizer was operated under a pressure of 3,000-8,000 psi with the interaction chamber placed in a water bath and maintained at a temperature of 4° C.-8° C., and the final pharmaceutical suspension (250 mg/ml) was obtained after homogenization.

The injection volume was reduced by 5 times, compared with the marketed product fulvestrant injection (50 mg/ml).

### Example 5 Preparation of 300 mg/ml Pharmaceutical Composition by Media Grinding

| Formula | Amount | |
|---|---|---|
| Fulvestrant | 90 | g |
| Polysorbate 80 | 2.5 | g |
| Sodium carboxymethylcellulose | 4.6 | g |
| Citric acid monohydrate | 5.0 | g |
| Sodium dihydrogen phosphate | 2.2 | g |
| Sodium hydroxide | 2.4 | g |

| | |
|---|---|
| Water for injection | Balance to 300 ml |

### Preparation Process:

(1) All the excipients were added to water, stirred and dissolved, and then fulvestrant was added and dispersed uniformly by preliminary shearing with a shearer at a revolving speed of 3,000 rpm, to obtain a primary pharmaceutical suspension; and
(2) The primary pharmaceutical suspension was subjected to media grinding using a sand mill at a revolving speed of 2,000-3,000 rpm, a peristaltic pump at a revolving speed of 50 rpm, and a stirrer at a stirring speed of 300 rpm, to obtain the final pharmaceutical suspension (300 mg/ml) after grinding.

The injection volume was reduced by 6 times, compared with the marketed product fulvestrant injection (50 mg/ml).

### Example 6 Preparation of 300 mg/ml Pharmaceutical Composition by Air Jet Pulverization

| Formula | Amount | |
|---|---|---|
| Fulvestrant | 90 | g |
| Polysorbate 20 | 2.5 | g |
| Polyethylene glycol 4000 | 46 | g |
| Anhydrous citric acid | 5.0 | g |
| Sodium dihydrogen phosphate | 2.2 | g |
| Sodium hydroxide | 2.4 | g |

| | |
|---|---|
| Water for injection | Balance to 300 ml |

### Preparation Process:

(1) Fulvestrant was weighted and air-jet pulverized under the pressure of 0.9 Mpa to obtain the active ingredient with the specific surface area of 0.958 m²/g.
(2) All the excipients were added to water, stirred and dissolved, and then micronized fulvestrant was added and dispersed uniformly by shearing with a shearer at a revolving speed of 3,000 rpm for 5-15 min, to obtain the final pharmaceutical suspension (300 mg/ml).

The injection volume was reduced by 6 times, compared with the marketed product fulvestrant injection (50 mg/ml).

### Example 7 Preparation of 400 mg/ml Pharmaceutical Composition by High-Speed Shearing

| Formula | Amount | |
|---|---|---|
| Fulvestrant | 120 | g |
| Polysorbate 80 | 3.2 | g |
| Sodium carboxymethylcellulose | 6.1 | g |
| Citric acid monohydrate | 2.4 | g |
| Sodium dihydrogen phosphate | 1.9 | g |
| Sodium hydroxide | 1.7 | g |

| | |
|---|---|
| Water for injection | Balance to 300 ml |

### Preparation Process:

(1) All the excipients were added to water, stirred and dissolved, and then fulvestrant was added and dispersed uniformly by further stirring with a stirrer at a speed of 1,000 rpm, to obtain a primary pharmaceutical suspension; and
(2) The primary pharmaceutical suspension was subjected to high-speed shearing with a shearer at a revolving speed of 10,000-30,000 rpm for 20-50 min, to obtain the final pharmaceutical suspension (400 mg/ml).

The injection volume was reduced by 8 times, compared with the marketed product fulvestrant injection (50 mg/ml).

### Example 8 Preparation of 400 mg/ml Pharmaceutical Composition by High-Pressure Homogenization

| Formula | Amount | |
|---|---|---|
| Fulvestrant | 120 | g |
| Polysorbate 60 | 3.2 | g |
| Polyethylene glycol 4000 | 24 | g |
| Tartaric acid | 2.4 | g |
| Disodium hydrogen phosphate | 1.9 | g |
| Sodium hydroxide | 1.7 | g |

| | |
|---|---|
| Water for injection | Balance to 300 ml |

### Preparation Process:

(1) All the excipients were added to water, stirred and dissolved, and then fulvestrant was added and dispersed uniformly by further stirring with a stirrer at a speed of 1,000 rpm, to obtain a primary pharmaceutical suspension; and
(2) The primary pharmaceutical suspension was homogenized for 1-6 min with a micro-jet high-pressure homogenizer that was operated under a pressure of 5,000-20,000 psi with the interaction chamber placed in a water bath and maintained at a temperature of 4° C.-8° C., and the final pharmaceutical suspension (400 mg/ml) was obtained after homogenization.

The injection volume was reduced by 8 times, compared with the marketed product fulvestrant injection (50 mg/ml).

### Example 9 Preparation of 500 mg/ml Pharmaceutical Composition by Media Grinding

| Formula | Amount | |
|---|---|---|
| Fulvestrant | 250 | g |
| Polysorbate 80 | 5.2 | g |
| Sodium carboxymethylcellulose | 12.8 | g |
| Citric acid monohydrate | 3.1 | g |
| Sodium dihydrogen phosphate | 3.1 | g |
| Sodium hydroxide | 2.8 | g |

| | |
|---|---|
| Water for injection | Balance to 500 ml |

### Preparation Process:

(1) All the excipients were added to water, stirred and dissolved, and then fulvestrant was added and dispersed uniformly by preliminary shearing with a shearer at a revolving speed of 3,000 rpm, to obtain a primary pharmaceutical suspension; and
(2) The primary pharmaceutical suspension was subjected to media grinding using a sand mill at a revolving speed of 2,000-4,000 rpm, a peristaltic pump at a revolving speed of 50 rpm, and a stirrer at a stirring speed of 300 rpm, to obtain the final pharmaceutical suspension (500 mg/ml) after grinding.

The injection volume was reduced by 10 times, compared with the marketed product fulvestrant injection (50 mg/ml).

### Example 10 Preparation of 500 mg/ml Pharmaceutical Composition by High-Pressure Homogenization

| Formula | Amount | |
|---|---|---|
| Fulvestrant | 250 | g |
| Polysorbate 80 | 5.2 | g |
| Sodium carboxymethylcellulose | 12.8 | g |
| Citric acid monohydrate | 3.1 | g |
| Sodium dihydrogen phosphate | 3.1 | g |
| Sodium hydroxide | 2.8 | g |

| | |
|---|---|
| Water for injection | Balance to 500 ml |

address can be associated with internet gateway router 512.

### Preparation Process:

(1) All the excipients were added to water, stirred and dissolved, and then fulvestrant was added and dispersed uniformly by further stirring with a stirrer at a speed of 1,000 rpm, to obtain a primary pharmaceutical suspension; and
(2) The primary pharmaceutical suspension was directly added to a micro-jet high-pressure homogenizer, and homogenized for 1-5 min under stirring with a glass rod to prevent drug precipitation; the micro-jet high-pressure homogenizer was operated under a pressure of 5,000-15,000 psi, and the final pharmaceutical suspension (500 mg/ml) was obtained after homogenization.

The injection volume was reduced by 10 times, compared with the marketed product fulvestrant injection (50 mg/ml).

### Example 11 Preparation of 550 mg/ml Pharmaceutical Composition by High-Pressure Homogenization

| Formula | Amount | |
|---|---|---|
| Fulvestrant | 275 | g |
| Polysorbate 60 | 5.7 | g |
| Polyethylene glycol 4000 | 14.1 | g |
| Tartaric acid | 3.4 | g |
| Disodium hydrogen phosphate | 3.4 | g |
| Sodium hydroxide | 3.1 | g |

| | |
|---|---|
| Water for injection | Balance to 500 ml |

### Preparation Process:

(1) All the excipients were added to water, stirred and dissolved, and then fulvestrant was added and dispersed uniformly by further stirring with a stirrer at a speed of 1,000 rpm/min, to obtain a primary pharmaceutical suspension; and
(2) The primary pharmaceutical suspension was homogenized for 1-6 min with a micro-jet high-pressure homogenizer that was operated under a pressure of 5,000-20,000 psi with the interaction chamber placed in a water bath and maintained at a temperature of 4° C.-8° C., and the final pharmaceutical suspension (400 mg/ml) was obtained after homogenization.

The injection volume was reduced by 11 times, compared with the marketed product fulvestrant injection (50 mg/ml)

Specific surface area determination for Examples 1-11:

TABLE 1
Average specific surface area (m²/g) of fulvestrant in the samples of Examples 1-11

| Examples | 1 | 2 | 3 | 4 | 5 | 6 | 7 | 8 | 9 | 10 | 11 | 12 |
|---|---|---|---|---|---|---|---|---|---|---|---|---|
| Average specific | 0.509 | 0.892 | 1.155 | 2.363 | 5.809 | 0.958 | 1.976 | 5.404 | 9.976 | 7.671 | 10.232 | 1.989 |
| surface area | | | | | | | | | | | | |

### Example 12 Preparation of Lyophilized Blocky Pharmaceutical Composition (200 mg/ml)

| Formula | Amount | |
|---|---|---|
| Fulvestrant | 100 | g |
| Polysorbate 20 | 3 | g |
| Polyethylene glycol 4000 | 24 | g |
| Citric acid | 3 | g |
| Sodium dihydrogen phosphate | 2.4 | g |
| Sodium hydroxide | 2.16 | g |

| | |
|---|---|
| Water for injection | Balance to 500 ml |

### Preparation Process:

(1) All the excipients were added to water, stirred and dissolved, and then fulvestrant was added and dispersed uniformly by preliminary stirring with a stirrer at a speed of 3,000 rpm, to obtain a primary pharmaceutical suspension;
(2) The primary pharmaceutical suspension was subjected to media grinding using a sand mill at a revolving speed of 2,000 rpm, a peristaltic pump at a revolving speed of 50 rpm, and a stirrer at a stirring speed of 300 rpm, to obtain the final pharmaceutical suspension (200 mg/ml) after grinding; and
(3) The final pharmaceutical suspension was filled, and pre-frozen, and then lyophilized for 48 h, to finally obtain the lyophilized blocky pharmaceutical composition.

### Example 13 Preparation of Lyophilized Blocky Pharmaceutical Composition (300 mg/ml)

| Formula | Amount | |
|---|---|---|
| Fulvestrant | 150 | g |
| Polysorbate 20 | 3 | g |
| Polyethylene glycol 4000 | 24 | g |
| Citric acid | 3 | g |
| Sodium dihydrogen phosphate | 2.4 | g |
| Sodium hydroxide | 2.16 | g |

| | |
|---|---|
| Water for injection | Balance to 500 ml |

### Preparation Process:

(1) All the excipients were added to water, stirred and dissolved, and then fulvestrant was added and dispersed uniformly by preliminary stirring with a stirrer at a speed of 3,000 rpm, to obtain a primary pharmaceutical suspension;
(2) The primary pharmaceutical suspension was subjected to media grinding using a sand mill at a revolving speed of 2,000 rpm, a peristaltic pump at a revolving speed of 50 rpm, and a stirrer at a stirring speed of 300 rpm, to obtain the final pharmaceutical suspension (300 mg/ml) after grinding; and
(3) The final pharmaceutical suspension was filled, and pre-frozen, and then lyophilized for 48 h, to finally obtain the lyophilized blocky pharmaceutical composition.

### Example 14 Preparation of Lyophilized Blocky Pharmaceutical Composition (400 mg/ml)

| Formula | Amount | |
|---|---|---|
| Fulvestrant | 200 | g |
| Polysorbate 20 | 3 | g |
| Polyethylene glycol 4000 | 24 | g |
| Citric acid | 3 | g |
| Sodium dihydrogen phosphate | 2.4 | g |
| Sodium hydroxide | 2.16 | g |

| | |
|---|---|
| Water for injection | Balance to 500 ml |

### Preparation Process:

(1) All the excipients were added to water, stirred and dissolved, and then fulvestrant was added and dispersed uniformly by preliminary stirring with a stirrer at a speed of 3,000 rpm, to obtain a primary pharmaceutical suspension;
(2) The primary pharmaceutical suspension was subjected to media grinding using a sand mill at a revolving speed of 2,000 rpm, a peristaltic pump at a revolving speed of 50 rpm, and a stirrer at a stirring speed of 300 rpm, to obtain the final pharmaceutical suspension (400 mg/ml) after grinding; and
(3) The final pharmaceutical suspension was filled, and pre-frozen, and then lyophilized for 48 h, to finally obtain the lyophilized blocky pharmaceutical composition.

### Example 15 Preparation of Lyophilized Blocky Pharmaceutical Composition (400 mg/ml)

| Formula | Amount | |
|---|---|---|
| Fulvestrant | 200 | g |
| Polysorbate 80 | 3 | g |
| Sodium carboxymethylcellulose | 1.6 | g |
| Mannitol | 10 | g |
| Sodium dihydrogen phosphate | 1.5 | g |
| Sodium hydroxide | 0.35 | g |

| | |
|---|---|
| Water for injection | Balance to 500 ml |

### Preparation Process:

(1) All the excipients were added to water, stirred and dissolved, and then fulvestrant was added and dispersed uniformly by preliminary stirring with a stirrer at a speed of 3,000 rpm, to obtain a primary pharmaceutical suspension;
(2) The primary pharmaceutical suspension was subjected to media grinding using a sand mill at a revolving speed of 2,000 rpm, a peristaltic pump at a revolving speed of 50 rpm, and a stirrer at a stirring speed of 300 rpm, to obtain the final pharmaceutical suspension (400 mg/ml) after grinding; and
(3) The final pharmaceutical suspension was filled, and pre-frozen, and then lyophilized for 48 h, to finally obtain the lyophilized blocky pharmaceutical composition.

### Example 16 Preparation of Pharmaceutical Suspension (200-400 mg/ml) Through Diluting Lyophilized Blocky Pharmaceutical Composition

| Formula | Amount | |
|---|---|---|
| Fulvestrant | 100 | g |
| Polysorbate 20 | 3 | g |
| Polyethylene glycol 4000 | 24 | g |
| Citric acid | 3 | g |
| Sodium dihydrogen phosphate | 2.4 | g |
| Sodium hydroxide | 2.16 | g |

| | |
|---|---|
| Water for injection | Balance to 667 ml |

### Preparation Process:

(1) All the excipients were added to water, stirred and dissolved, and then fulvestrant was added and dispersed uniformly by preliminary stirring with a stirrer at a speed of 3,000 rpm, to obtain a primary pharmaceutical suspension;
(2) The primary pharmaceutical suspension was subjected to media grinding using a sand mill at a revolving speed of 2,000 rpm, a peristaltic pump at a revolving speed of 50 rpm, and a stirrer at a stirring speed of 300 rpm, to obtain an unfrozen pharmaceutical suspension (150 mg/ml) after grinding;
(3) The pharmaceutical suspension was filled into a vial, pre-frozen, and then lyophilized for 48 h, to finally obtain a lyophilized blocky pharmaceutical composition; and
(4) The lyophilized blocky pharmaceutical composition was diluted with an appropriate amount of water for injection to provide a suspension with a fulvestrant concentration of 200 mg/ml, or 250 mg/ml, or 300 mg/ml, or 350 mg/ml, or 400 mg/ml.

### Example 17 Pharmacokinetic Study

Pharmacokinetic evaluation of pharmaceutical compositions prepared in Examples 1, 2, 3, 4, 5, 10, and 11 was carried out using beagle dogs as the animal model, along with the administration of the marketed preparation fulvestrant injection (Faslodex) at a dosage of 50 mg/kg. Blood samples were collected from the forelimb vein at different time points after administration. Faslodex had the steady-state plasma concentration (effective concentration) in the range of 6 ng/ml to 50 ng/ml after several consecutive administrations.

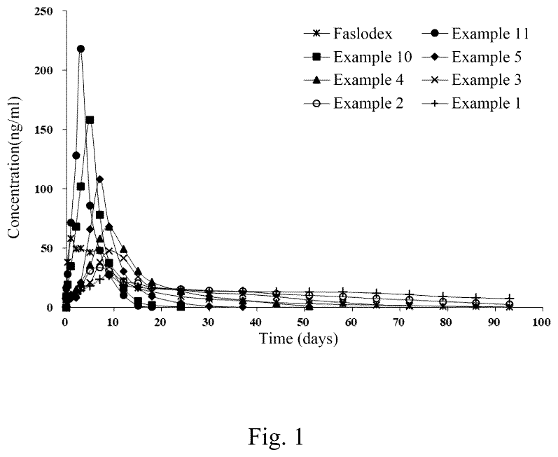

The single-dose pharmacokinetic profiles were shown in FIG. 1. The pharmaceutical compositions in Examples 10 and 11 had larger specific surface area, and exhibited a burst release of the drug and a shorter effective concentration maintenance time for the same administration amount at the in vivo drug concentration of about 6 ng/ml. Faslodex can be sustainedly released for about 4 weeks, and is administered about once a month. Compared with the commercially available drug “Faslodex”, the fulvestrant suspension injection sample in Example 1 of the present disclosure was released slowly in the dogs, which could be maintained for about 12 weeks, and could be administered once in about 3 months; the fulvestrant suspension injection sample in Example 2 of the present disclosure was released slowly in the dogs, which could be maintained for about 8 weeks, and could be administered once in about 2 months; the fulvestrant suspension injection sample in Example 3 of the present disclosure was released slowly in the dogs, which could be maintained for about 6 weeks, and could be administered once in about 1.5 months; the fulvestrant suspension injection sample in Example 4 of the present disclosure was released slowly in the dogs, which could be maintained for about 4 weeks, and could be administered about once a month; and the fulvestrant suspension injection sample in Example 5 of the present disclosure was released quickly in the dogs, which could be maintained for about 2 weeks, and could be administered once in about 0.5 month.

In summary, the pharmaceutical composition of the present disclosure having the specific surface area in the above-mentioned range not only increased the drug concentration, but also had a significant sustained release effect. The pharmaceutical composition of the present disclosure had a much smaller range of fluctuations in drug concentration, and maintained the drug release for a period of time of 0.5 month to 3 months. Furthermore, the pharmaceutical composition of the present disclosure had greater clinical advantages because of reduced injection volume and reduced administration frequency.

### Example 18 Stability Study

In order to evaluate the stability of drug solutions with different particle sizes, the samples in Examples 2, 3, 6, 7, 8, and 9 were placed at 25° C., 30° C., 40° C., and 2-8° C., to determine the specific surface area and perform appearance observation at different time points. The appearance observation result showed that all the placed samples were white suspensions. The specific surface area determination results showed that the samples remained stable at different temperatures and could be placed at room temperature for at least 12 months, with no change in the specific surface area of fulvestrant during the placement period. The samples in Examples 2, 3, 6, 7, 8, and 9 showed a significant increase in the concentrations of fulvestrant compared with the fulvestrant preparations in the prior art, indicating that the samples had better stability.

TABLE 2
Specific surface area (m²/g) of fulvestrant in samples of
Examples 2, 3, 6, 7, 8, and 9 at different time points

| Time | Temperature | | 0 d | 5 d | 10 d | 15 d | 1 M | 2 M | 3 M | 6 M | 9 M | 12 M |
|---|---|---|---|---|---|---|---|---|---|---|---|---|
| Example 2 | 2-8° | C. | 0.892 | 0.873 | 0.901 | 0.882 | 0.876 | 0.867 | 0.861 | 0.859 | 0.868 | 0.853 |
| | 25° | C. | | 0.897 | 0.908 | 0.886 | 0.895 | 0.881 | 0.877 | 0.868 | 0.881 | 0.869 |
| | 30° | C. | | 0.884 | 0.903 | 0.873 | 0.886 | 0.873 | 0.867 | 0.859 | 0.861 | 0.858 |
| | 40° | C. | | 0.878 | 0.870 | 0.869 | 0.852 | 0.843 | 0.849 | \ | \ | \ |
| Example 3 | 2-8° | C. | 1.155 | 1.163 | 1.153 | 1.157 | 1.149 | 1.146 | 1.157 | 1.140 | 1.168 | 1.144 |
| | 25° | C. | | 1.157 | 1.162 | 1.152 | 1.143 | 1.140 | 1.132 | 1.137 | 1.129 | 1.133 |
| | 30° | C. | | 1.162 | 1.158 | 1.149 | 1.139 | 1.149 | 1.130 | 1.128 | 1.121 | 1.127 |
| | 40° | C. | | 1.150 | 1.148 | 1.137 | 1.135 | 1.121 | 1.117 | \ | \ | \ |
| Example 6 | 2-8° | C. | 0.958 | 0.917 | 0.938 | 0.918 | 0.969 | 0.979 | 0.969 | 0.932 | 0.968 | 0.954 |
| | 25° | C. | | 0.901 | 0.918 | 0.952 | 0.997 | 0.957 | 0.955 | 0.907 | 0.912 | 0.935 |
| | 30° | C. | | 0.916 | 0.907 | 0.939 | 0.902 | 0.951 | 0.996 | 0.979 | 0.937 | 0.971 |
| | 40° | C. | | 0.945 | 0.981 | 0.924 | 0.954 | 0.978 | 0.969 | \ | \ | \ |
| Example 7 | 2-8° | C. | 1.976 | 1.965 | 1.968 | 1.944 | 1.912 | 1.912 | 1.903 | 1.923 | 1.976 | 1.982 |
| | 25° | C. | | 1.911 | 1.933 | 1.980 | 1.964 | 1.994 | 1.948 | 1.979 | 1.983 | 1.923 |
| | 30° | C. | | 1.957 | 1.948 | 1.943 | 1.926 | 1.983 | 1.946 | 1.938 | 1.955 | 1.984 |
| | 40° | C. | | 1.951 | 1.939 | 2.403 | 1.931 | 1.981 | 1.984 | \ | \ | \ |
| Example 8 | 2-8° | C. | 5.404 | 5.459 | 5.427 | 5.465 | 5.498 | 5.412 | 5.479 | 5.423 | 5.449 | 5.479 |
| | 25° | C. | | 5.368 | 5.406 | 5.420 | 5.416 | 5.458 | 5.423 | 5.381 | 5.422 | 5.453 |
| | 30° | C. | | 5.412 | 5.411 | 5.386 | 5.445 | 5.455 | 5.464 | 5.499 | 5.468 | 5.497 |
| | 40° | C. | | 5.474 | 5.401 | 5.481 | 5.428 | 5.485 | 5.482 | \ | \ | \ |
| Example 9 | 2-8° | C. | 9.976 | 9.922 | 9.973 | 9.994 | 9.987 | 9.986 | 9.967 | 9.996 | 10.052 | 9.983 |
| | 25° | C. | | 9.971 | 10.076 | 9.957 | 9.939 | 9.913 | 9.940 | 10.064 | 9.993 | 10.047 |
| | 30° | C. | | 9.935 | 9.917 | 9.964 | 9.921 | 9.939 | 9.909 | 9.935 | 9.930 | 9.890 |
| | 40° | C. | | 9.992 | 9.909 | 9.886 | 9.901 | 10.046 | 10.116 | \ | \ | \ |

What is claimed is:

1. A pharmaceutical composition comprising an active ingredient, an excipient, and an aqueous carrier, and free of ethanol and an oily matrix, wherein the active ingredient is fulvestrant or an ester derivative of fulvestrant, the excipient comprises one or more selected from the group consisting of a wetting agent, a suspending agent, a buffer agent, and an osmotic pressure regulator, and a concentration of the active ingredient is from 150 mg/ml to 550 mg/ml, preferably from 200 mg/ml to 400 mg/ml, calculated on the basis of fulvestrant itself.

2. The pharmaceutical composition according to claim 1, wherein the pharmaceutical composition has a dosing volume of from 2 ml to 8 ml, preferably from 3 ml to 6 ml.

3. The pharmaceutical composition according to claim 1, wherein the pharmaceutical composition comprises the active ingredient and the excipient at the following contents in weight percentage (w/w):

| | |
|---|---|
| active ingredient | 15-55% |
| wetting agent | 0.1-2% |
| suspending agent | 0.1-20% |
| buffer agent | 0.1-10% |
| osmotic pressure regulator | 0.1-15% |
| aqueous carrier | balance to 100%, |

and preferably, the pharmaceutical composition comprises the active ingredient and the excipient at the following contents in weight percentage (w/w).

| | |
|---|---|
| active ingredient | 20-40% |
| wetting agent | 0.3-1.5% |
| suspending agent | 0.3-16% |
| buffer agent | 0.3-5% |
| osmotic pressure regulator | 0.3-10% |
| aqueous carrier | balance to 100%. |

4. The pharmaceutical composition according to claim 1, wherein the aqueous carrier is selected from water for injection, glucose injection, or normal saline; and preferably, the aqueous carrier is water for injection.

5. The pharmaceutical composition according to claim 1, wherein the excipient comprises a wetting agent, a suspending agent, a buffer agent, and an osmotic pressure regulator.

6. The pharmaceutical composition according to claim 1, wherein the wetting agent is selected from one or more of polysorbate 20, polysorbate 60, polysorbate 80, lecithin, sodium deoxycholate, poloxamer, and sodium lauryl sulfate;
the suspending agent is selected from one or more of sodium carboxymethylcellulose, hypromellose, hydroxypropylcellulose, povidone, polyethylene glycol 4000, and polyethylene glycol 6000;
the buffer agent is selected from one or more of phosphate, citric acid or citrate, tartaric acid or tartrate, acetate, HEPES, Tirs, and sodium hydroxide; and/or
the osmotic pressure regulator is selected from one or more of sucrose, mannitol, trehalose, sodium chloride, phosphate, citric acid or citrate, and tartaric acid or tartrate.

7. The pharmaceutical composition according to claim 3, wherein the wetting agent is selected from one or more of polysorbate 20, polysorbate 60, polysorbate 80, lecithin, sodium deoxycholate, poloxamer, and sodium lauryl sulfate;
the suspending agent is selected from one or more of sodium carboxymethylcellulose, hypromellose, hydroxypropylcellulose, povidone, polyethylene glycol 4000, and polyethylene glycol 6000;
the buffer agent is selected from one or more of phosphate, citric acid or citrate, tartaric acid or tartrate, acetate, HEPES, Tirs, and sodium hydroxide; and/or
the osmotic pressure regulator is selected from one or more of sucrose, mannitol, trehalose, sodium chloride, phosphate, citric acid or citrate, and tartaric acid or tartrate.

8. The pharmaceutical composition according to claim 1, wherein the active ingredient has an average specific surface area of 0.5-5.8 m²/g; and
preferably, the active ingredient has an average specific surface area of 0.5-2.5 m²/g.

9. The pharmaceutical composition according to claim 3, wherein the active ingredient has an average specific surface area of 0.5-5.8 m²/g; and
preferably, the active ingredient has an average specific surface area of 0.5-2.5 m²/g.

10. The pharmaceutical composition according to claim 6, wherein the active ingredient has an average specific surface area of 0.5-5.8 m²/g; and
preferably, the active ingredient has an average specific surface area of 0.5-2.5 m²/g.

11. The pharmaceutical composition according to claim 7, wherein the active ingredient has an average specific surface area of 0.5-5.8 m²/g; and
preferably, the active ingredient has an average specific surface area of 0.5-2.5 m²/g.

12. The pharmaceutical composition according to claim 1, wherein the pharmaceutical composition is in the form of a suspension; or the pharmaceutical composition is in the form of a blocky or powdered composition converted by lyophilization, and the blocky or powdered composition is reconstituted into a suspension using water for injection, glucose injection, or normal saline for clinical use.

13. The pharmaceutical composition according to claim 1, wherein the pharmaceutical composition comprises the following components at contents of.

| Formula | Amount | |
|---|---|---|
| fulvestrant | 100 | g |
| polysorbate 20 | 3 | g |
| polyethylene glycol 4000 | 24 | g |
| citric acid | 3 | g |
| sodium dihydrogen phosphate | 2.4 | g |
| sodium hydroxide | 2.16 | g |
| | |
|---|---|
| water for injection | balance to 500 ml or 400 ml or 333 ml |
| | or 286 ml or 250 ml. |

14. The pharmaceutical composition according to claim 1, wherein the active ingredient is dispersed in the aqueous carrier, and the excipient is dissolved in the aqueous carrier.

15. A method for preparing the pharmaceutical composition according to claim 1, comprising steps of:
method I-preparation of a pharmaceutical suspension by air jet pulverization:
(1) air jet pulverizing an active ingredient to a desired average specific surface area to obtain a micronized active ingredient, and
(2) dissolving an excipient in an aqueous carrier, adding the micronized active ingredient, and dispersing the components uniformly by stirring or shearing, to obtain the pharmaceutical suspension;
wherein the average specific surface area in step (1) is 0.5-5.8 m²/g, or
method II-preparation of a pharmaceutical suspension by high-speed shearing, media grinding, or high-pressure homogenization:
(1) dissolving an excipient in an aqueous carrier, then adding an active ingredient, and dispersing the active ingredient uniformly by stirring or preliminary shearing, to obtain a primary pharmaceutical suspension, and
(2) subjecting the primary pharmaceutical suspension to high-speed shearing, media grinding, or high-pressure homogenization, so that the active ingredient has a specific surface area of 0.5-5.8 m²/g, thereby obtaining the pharmaceutical composition in the form of the suspension.

16. The preparation method according to claim 15, further comprising steps of:
filling the pharmaceutical composition in the form of the suspension to obtain a pharmaceutical product in the form of a liquid; or
lyophilizing the pharmaceutical composition in the form of the suspension to obtain a lyophilized product of fulvestrant in the form of a block or powder, wherein the lyophilized product of fulvestrant is reconstituted with water for injection, glucose injection, or normal saline into a suspension with a fulvestrant concentration of 150 mg/ml-550 mg/ml for clinical use, and preferably, the lyophilized product of fulvestrant is reconstituted with water for injection into a suspension with a fulvestrant concentration of 200 mg/ml, or 250 mg/ml, or 300 mg/ml, or 350 mg/ml, or 400 mg/ml for clinical use.

17. A method for treating advanced or metastatic breast cancer in a patient in need thereof, comprising administering the pharmaceutical composition according to claim 1 to the patient.
